(19) United States
(12) Patent Application Publication
(10) Pub. No.: US 2012/0294808 A1
(43) Pub. Date: Nov. 22, 2012

(54) Fluorescent Analogs of Neurotransmitters, Compositions Containing the Same and Methods of Using the Same

(75) Inventor: James N. Wilson, Palmetto Bay, FL (US)

(73) Assignee: UNIVERSITY OF MIAMI, Miami, FL (US)

(21) Appl. No.: 13/508,580

(22) PCT Filed: Nov. 9, 2010
(86) PCT No.: PCT/US10/55912

§ 371 Date: May 8, 2012

Related U.S. Application Data
(60) Provisional application No. 61/259,434, filed on Nov. 9, 2009.

(51) Int. Cl. A61K 49/00; G01N 21/64; C07D 221/14; C07D 513/04; C07D 215/227; C07D 213/30

(52) U.S. Cl. 424/9.6; 546/157; 546/344; 546/100; 544/34; 435/29

(57) ABSTRACT

A method of imaging using selective fluorescent emitters and compositions for imaging are described. The method can include contacting a specimen with a composition comprising at least one selective fluorescent emitter of Formulas I through IX and irradiating the specimen with an excitation wavelength. The fluorescence emitted by the selective fluorescent emitter can be detected to generate an image. Formulas I through IX are:

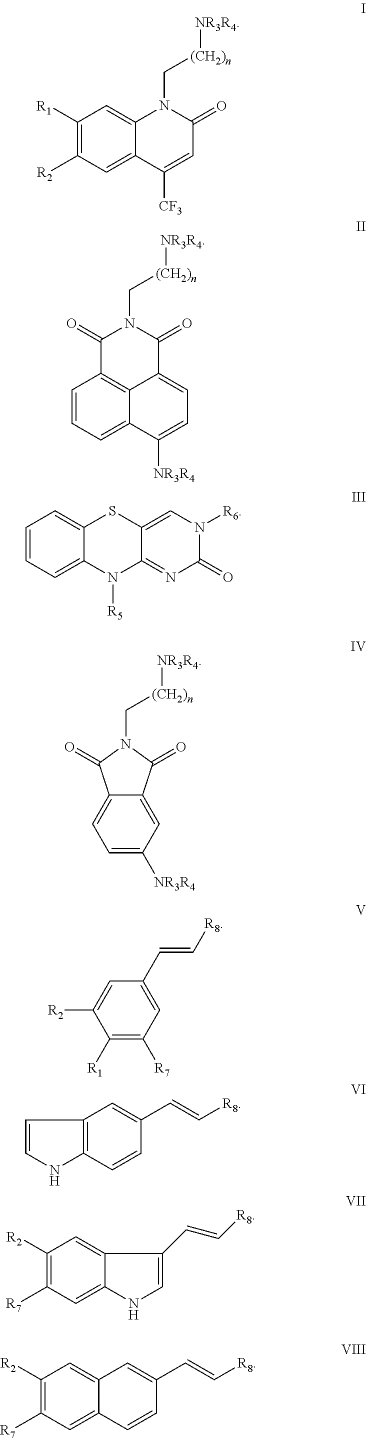

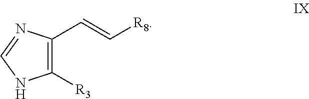

wherein: R₁=OR₄ or NR₃R₄; R₂=OR₄, NR₃R₄, Cl, F or H; R₃=H, CH₃, C₂H₅, C₃H₇ or C₄H₉; R₄=H, CH₃, C₂H₅, C₃H₇ or C₄H₉; R₅=H or CH₂(CH₂)ₙNR₃R₄; R₆=H or CH₂(CH₂)ₙNR₃R₄; R₇=OR₄, NR₃R₄, Cl, F or H; R₈=N-methylpyridinium or N-methyl-1,2,3,6-tetrahydropyridine; and n=1, 2 or 3.

### CROSS REFERENCE TO RELATED APPLICATIONS

This Application claims priority to U.S. Provisional Patent Application No. 61/259,434, “Fluorescent Analogs of Serotonin, Compositions Containing the Same and Methods of Using the Same,” entitled Filed Nov. 9, 2009, the entirety of which is incorporated herein by reference.

### FIELD OF THE INVENTION

The present invention is generally directed toward fluorescent analogs of neurotransmitters, compositions containing the same and methods of imaging using the same.

### BACKGROUND OF THE INVENTION

Since the development of the epifluorescent microscope nearly a century ago, fluorescent probes have played a central role in imaging and biotechnology applications. In most, but not all cases, a fluorescent label is covalently appended to a biomolecule that imparts specificity for a target. Notable examples of this construct include fluorescently labeled antibodies used in immunohistochemical assays and fluorescent fusion proteins which are invaluable reporters of protein expression or localization. Fluorescent analogs of biomolecules offer an alternative strategy to fluorescently labeled biomolecules. By employing an inherently fluorescent structure that closely mimics the native molecule, it is possible to avoid additional steric bulk, changes in shape, or ionic character that some fluorophores impart. Fluorescent nucleobases have been successfully demonstrated in structural studies and enzymatic assays. In order to be useful as a selective fluorescent emitter, a compound must (i) interact selectively with target components of the nervous system, e.g., transporters, receptors, enzymes, cells, etc., (ii) emit radiation at a wavelength that avoids background noise when irradiated by an excitation wavelength; and (iii) emit enough radiation that it is possible to obtain useful images. Because the properties necessary for a useful selective fluorescent emitter are difficult to predict, identifying compounds useful as selective fluorescent emitters is far from trivial.

### SUMMARY OF THE INVENTION

In one embodiment, the invention is drawn to a method of imaging that includes contacting a specimen with a composition comprising a selective fluorescent emitter selected from Formulas I through IX, and irradiating the specimen with an excitation wavelength. Formulas I through IX are:

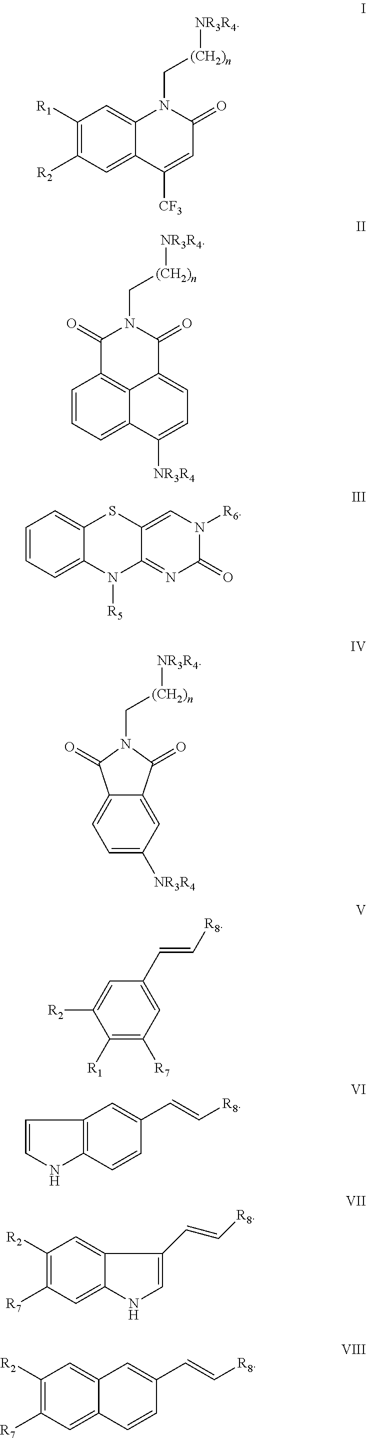

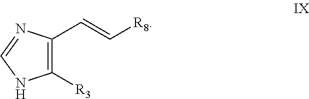

wherein:
R₁=OR₄ or NR₃R₄;
R₂=OR₄, NR₃R₄, Cl, F or H;
R₃=H, CH₃, C₂H₅, C₃H₇ or C₄H₉;
R₄=H, CH₃, C₂H₅, C₃H₇ or C₄H₉;
R₅=H or CH₂(CH₂)ₙNR₃R₄;
R₆=H or CH₂(CH₂)ₙNR₃R₄;
R₇=OR₄, NR₃R₄, Cl, F or H;
R₈=N-methylpyridinium or N-methyl-1,2,3,6-tetrahydropyridine; and
n=1, 2 or 3.

The method can include detecting fluorescence from the treated specimen to generate an image. The detecting step comprises obtaining a camera imaging of the specimen. The detecting step can include filtering out electromagnetic radiation below 400 nm.

The selective fluorescent emitter emits fluorescence at a wavelength above 400 nm when irradiated at the excitation wavelength. The excitation wavelength can include electromagnetic radiation having a wavelength below 400 nm.

The irradiating step comprises irradiating with a monochromatic electromagnetic radiation source. The monochromatic electromagnetic radiation source can include radiation having a wavelength below 400 nm. The monochromatic electromagnetic radiation source can be a diode laser.

The selective fluorescent emitter can include a compound selected from the group consisting of Formulas I, II, III and IV. In such embodiments, the remaining constituents can be at least one of the following:

R₃=H or CH₃ and R₄=H or CH₃;

R₃ and R₄ can be H;

R₂=H, R₅=H and R₆=CH₂(CH₂)ₙNR₃R₄;

In other exemplary methods, the selective fluorescent emitter can include a compound selected from the group consisting of Formulas V, VI, VII, VIII and IX. In such methods, exemplary compounds of Formulas V, VI, VII, VIII and IX can be at least one of the following:

R₁=OH, OCH₃, NH₂, NHCH₃ or N(CH₃)₂, and R₃=H or CH₃;

A compound of Formula V, where R₁=OH or OCH₃ and R₂=R₇=H.

A compound of Formula VII or VIII, where R₇=OH, OCH₃, NH₂, NHCH₃ or N(CH₃)₂ and R₂=H;

A compound of Formula VII or VIII, where R₂=H and R₇=OH, OCH₃, or H;

A compound of Formula IX, where R₃=H or CH₃.

The selective fluorescent emitter can include at least one of the following compounds:

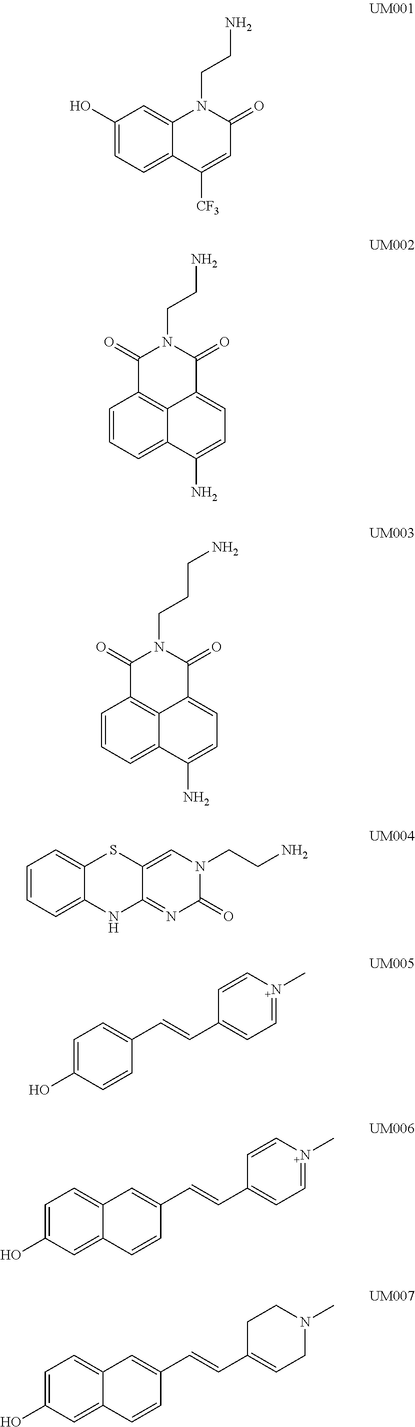

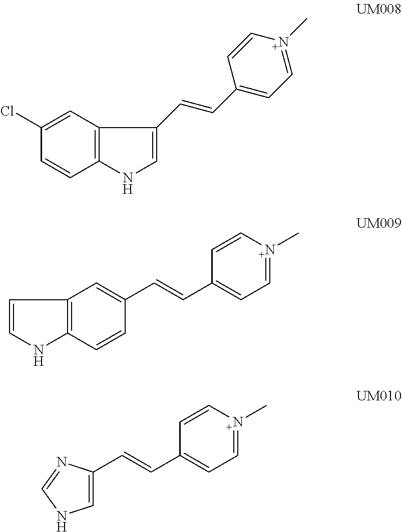

The method can include compositions that include a first selective fluorescent emitter selected from a first one of Formulas I through IX, and a second selective fluorescent emitter selected from a different one of Formulas I through IX. The first and second selective fluorescent emitter can fluoresce at wavelengths above 400 nm when irradiated by excitation radiation having a wavelength below 400 nm, and the first and said second selective fluorescent emitters fluoresce at different wavelengths.

The invention is also drawn to compositions that include at least one selective fluorescent emitter selected from Formulas I through IX as described herein. The composition can also include a solvent selected from the group consisting of an organic solvent, an aqueous solvent, or both. A concentration of the selective fluorescent emitter in the composition can be 1 nM to 10 M. The composition can be that of any of the compositions described in herein.

The invention is also drawn to a compound according to one of Formulas I through IX. The compounds can be any of the compounds of Formulas I through IX described herein, including:

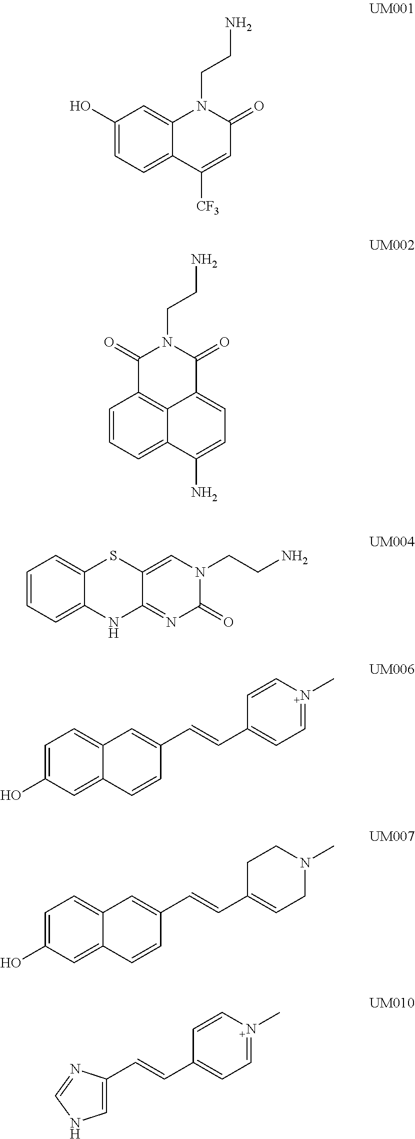

These and other embodiments are described in more detail below.

### BRIEF DESCRIPTION OF THE DRAWINGS

A fuller understanding of the present invention and the features and benefits thereof will be obtained upon review of the following detailed description together with the accompanying drawings, in which:

FIG. 1 is a graph of absorbance (Abs), excitation (Ex) and emission (Em) spectra of compound 1 compared with serotonin (5HT).

FIG. 2 is a graph of a microwell plate assay demonstrating the affinity of compounds 1 and 4 for cultured neurons and astroglia isolated from E14 chick brainstems.

FIGS. 3A-H are images taken when e14 chick brainstem cells were exposed to compound 1, where (A) is a phase contrast image of the viewing area; (B) is a control image before addition of 1 showing minimal cellular autofluorescence; (C) is a view of silhouetted cells as the fluorophore solution is added; (D) is 90 sec after addition, showing virtually all healthy cells have accumulated the probe; and (E)-(H) show selected z-stack sections (9 μm top to bottom) showing internalization of the fluorophore.

FIGS. 4A & B are plots of the absorption and emission spectra, respectively, for several potential selective fluorescent emitters.

FIG. 5 is a table showing whether each of 32 combinations of components have accumulated in dissociated chick brain cultures and fluoresce when irradiated at four different wavelengths (365 nm—top left, 405 nm—top right, 450 nm—bottom left, and 500 nm—bottom right), with black indicating no emission.

FIG. 6 is a chart showing fluorescent intensity for select compounds exposed to e19 chick whole brain cultures both with and without indatraline, a monoamine reuptake inhibitor.

FIG. 7 is a comparison of the behavior of compounds 13A and 14A toward HEK293 cells, where FIG. 7A shows fluorescent microscopy results 10 seconds after the introduction of 14A; FIG. 7B shows fluorescent microscopy results 150 second after the introduction of 14A;

FIG. 7C shows fluorescent microscopy results 10 seconds after the introduction of 13A; FIG. 7D shows fluorescent microscopy results 150 second after the introduction of 13A; and FIGS. 7E & F show intensity v. time plots for 14A and 13A, respectively.

FIG. 8 shows confocal microscopy images of live e19 chick brain explant treated with 13A and 14A.

FIG. 9 shows an ex vivo assessment of 14A uptake in the presence and absence of desipramine, a norepinephine reupdate inhibitor, where FIG. 9A is compound 14A alone, FIG. 9B is compound 14A after treatment with desipramine, FIG. 9C is a histogram of pixel intensity for 14A both with and without desipramine, and FIG. 9D is mean intensity of 14A both with and without desipramine.

### DETAILED DESCRIPTION

The invention is drawn to a variety of compounds, compositions and methods that are useful for research in a broad range of areas, including biotechnology, pharmaceuticals and neuroscience. Disclosed are selective fluorescent emitters that interact with cells, such as neurons and astroglia, in a manner analogous to neurotransmitters, such as serotonin, dopamine and norepinephrine. Using these selective fluorescent emitters it is possible to obtain images that help understand how these neurotransmitters interactions with the cells in the presence or absence of additional compounds, such as pharmaceuticals. For example, the inventive fluorescent emitters, compositions and methods can be used to screen whether a potential pharmaceutical compound would be effective as a serotonin reuptake inhibitor (SRI), a serotonin transporter (SERT), a dopamine transporter (DAT), a norepinephrine transporter (NET), etc. Similarly, the fluorescent emitters, compositions and methods described herein can be used to determine whether a modified cell has improved interactions with serotonin, dopamine and norepinephrine.

The method of imaging described herein can include contacting a specimen with a composition comprising a selective fluorescent emitter selected from Formulas I through IX. The treated specimen can then be irradiated with an excitation wavelength (λ_{EX}). The method can also include detecting fluorescence from the treated specimen to generate an image.

The detecting step can include obtaining a camera image of the treated specimen. For example, the fluorescence can be detected using a CCD camera, photomultiplier tube, photodiode or other similar devices. The image can be captured using any known picture taking technology, but is preferably captured digitally for further image processing. The detection device can be optically coupled to an epifluorescence microscope, a confocal microscope or other similar optical detection device. An exemplary microscopy technique includes fluorescence lifetime imaging (FLIM).

The capturing step can include the use of one or more optical filters, such as lenses, for filtering out background noise, such as electromagnetic radiation below 400 nm or even below 410 nm. Similarly, filters can use utilized to eliminate electromagnetic radiation below 400 nm, below 410 nm or below the excitation wavelength (λ_{EX}). The optical filters(s) can be absorptive filters, dichroic filters, or both.

Many biological materials that could be present in the specimen emit electromagnetic radiation when irradiated; however, much of this “ambient radiation” has a wavelength below 400 nm. Thus, the selective fluorescent emitters described herein, preferably emit fluorescence at an emission wavelength (λ_{EM}) above 400 nm when irradiated at the excitation wavelength. The wavelength emitted from the selective fluorescent emitter can range between 400 and 800 nm. The emission wavelength can be between 400 and 500 nm.

Where filters are used to eliminate radiation below the excitation wavelength (λ_{EX}), reflected radiation at the excitation wavelength (λ_{EX}) can be filtered out and the emitted radiation wavelength (λ_{EM}) detected. In such exemplary methods, the excitation wavelength will be filtered out along with the “ambient radiation” and will not interfere with the images.

The irradiating step can include irradiating with a monochromatic electromagnetic radiation source, such as a monochromatic electromagnetic radiation source emitting radiation at wavelength below 400 nm. The monochromatic electromagnetic radiation source can be a diode laser.

The specimen can be cells. The contacting step can include contacting the specimen with a test compound. For example, the specimen can be cells that have been treated with a test compound that may have an effect on interactions between the cells and a neurotransmitter. Exemplary test compounds include potential or existing pharmaceutical or other controlled or uncontrolled substance. Exemplary test compounds include serotonin reuptake inhibitors (SRI), serotonin transporters (SERT), dopamine transporters (DAT), dopamine reuptake inhibitors (DRI), norepinephrine transporters (NET), and norepinephrine reuptake inhibitors (NRI). Alternately, the specimen can be contacted with both the fluorescent probe and the test compound simultaneously or the specimen can be treated with the fluorescent probe prior to being contacted with the test compound. The specimen will generally include target cells, such as brain tissue or other tissue from the nervous system.

The specimen can also be modified cells, such as a genetically modified cell line. In such cases, the modified cells can be contacted with the fluorescent probe, a test compound, or both, and then be imaged. For example, an investigator could use the method described herein to determine if modified cells interact with neurotransmitters, such as serotonin, dopamine, and/or norepinephrine differently than unmodified cells.

Regardless of the order of treatment and the specimen type, the results can be compared with a control. Examples of controls will be apparent; however, examples include cells that are not treated with the test compound (e.g., serotonin reuptake inhibitors, serotonin transporters, dopamine transporters, and norepinephrine transporters, etc.) or an unmodified cell line.

The irradiation step can include using a monochromatic electromagnetic radiation source, such as a diode laser. The excitation wavelength can be above 400 nm, below 800 nm, or both. The excitation wavelength can range from 400 to 500 nm. The excitation wavelength can range from 400-450 nm.

The invention is also drawn to compositions, which can be used during the contacting step of the inventive method described herein. The compositions can include at least one selective fluorescent emitter of Formulas I through IX. Formulas I through IX are defined as follows:

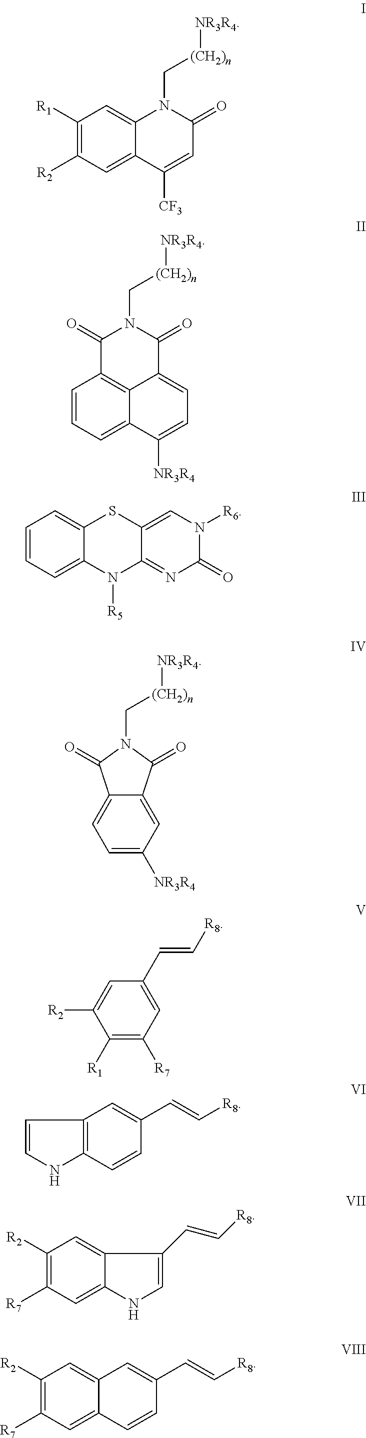

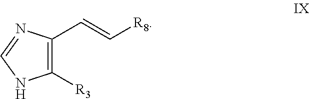

wherein
R₁=OR₄ or NR₃R₄;
R₂=OR₄, NR₃R₄, Cl, F or H;
R₃=H, CH₃, C₂H₅, C₃H₇ or C₄H₉;
R₄=H, CH₃, C₂H₅, C₃H₇ or C₄H₉;
R₅=H or CH₂(CH₂)ₙNR₃R₄;
R₆=H or CH₂(CH₂)ₙNR₃R₄;
R₇=OR₄, NR₃R₄, Cl, F or H;
R₈=N-methylpyridinium or N-methyl-1,2,3,6-tetrahydropyridine; and
n=1, 2 or 3.

As used herein, the ligands of R₈ have the following structures:

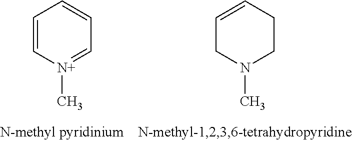

The bonds from the R₈ ligand to the remainder of the selective fluorescent emitter can be formed at the position opposite the nitrogen atom, i.e., position 4 if the nitrogen atom considered position 1. A synthesis for forming compounds including the ligands according to R₈ can be found in Example 2, below.

In some exemplary selective fluorescent emitters, n can be 1 or 2. The compositions described herein can include two, three, four or more compounds according to Formulas I-IX.

As will be understood, during the chemical synthesis, purification, etc., it may be possible to select a specific ion. The compounds of Formulas I-IV can generally be produced without a counter ion, while compounds of Formula V-IX must have a counter ion.

In compositions containing compounds of Formulas I, II, III and/or IV, the selective fluorescent emitters of Formulas I, II, III and/or IV can be present as the protonated ammonium salt with a counter ion of chloride, bromide, iodide, acetate or other anion. Exemplary compounds of Formulas I, II, III and IV, including compounds UM001 through UM004.

In compositions containing compounds of Formulas V, VI, VII, VIII and/or IX, the selective fluorescent emitters of Formulas V, VI, VII, VIII and/or IX can be present with a counter ion of chloride, bromide, iodide, acetate or other anion. Exemplary compounds of Formulas V, VI, VII, VIII and IX, including compounds UM005 through UM010.

The composition can include a selective fluorescent emitter selected from the group consisting of Formulas I, II, III and IV, where R₃=H or CH₃ and R₄=H or CH₃. Similarly, the composition can include a selective fluorescent emitter selected from the group consisting of Formulas I, II, III and IV, where R₃=R₄=H. In addition, the composition can include a selective fluorescent emitter selected from the group consisting of Formulas I, II, III and IV, where R₂=H, R₅=H and R₆=CH₂(CH₂)ₙNR₃R₄. In some examples, n can be 1 or 2.

The composition can also include a selective fluorescent emitter selected from the compounds according to Formulas V, VI, VII, VIII and IX, where R₁=OH, OCH₃, NH₂, NHCH₃ or N(CH₃)₂, and R₃=H or CH₃. Similarly, the composition can include one, two, three, or more of the following:

i. a compound of Formula V, wherein R₁=OH or OCH₃ and R₂=R₇=H;

ii. a compound of Formula VII or VIII, wherein R₇=OH, OCH₃, NH₂, NHCH₃ or N(CH₃)₂ and R₂=H;

iii. a compound of Formula VII or VIII, wherein R₂=H and R₇=OH, OCH₃, or H; or

iv. a compound of Formula IX, wherein R₃=H or CH₃.

Particular selective fluorescent emitters of interest include the following:

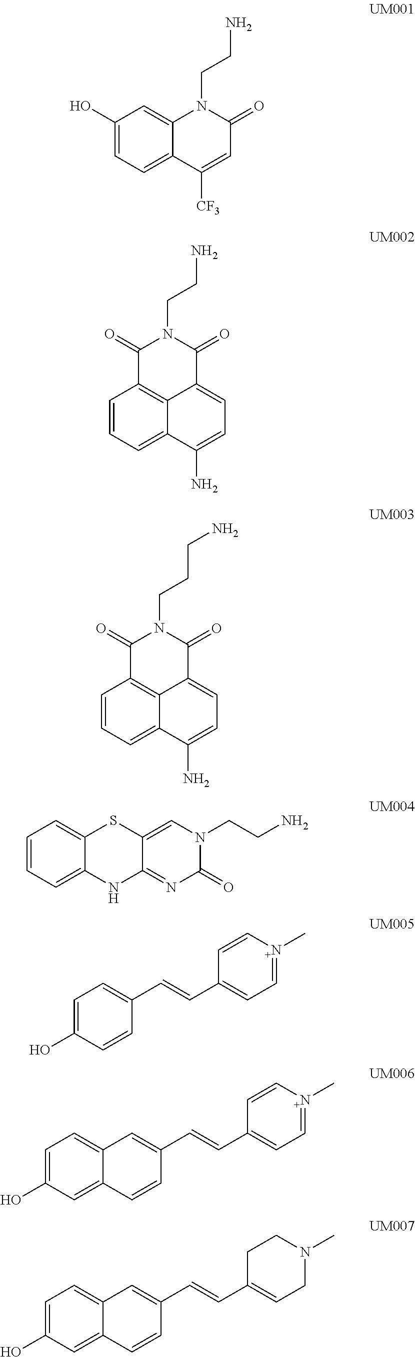

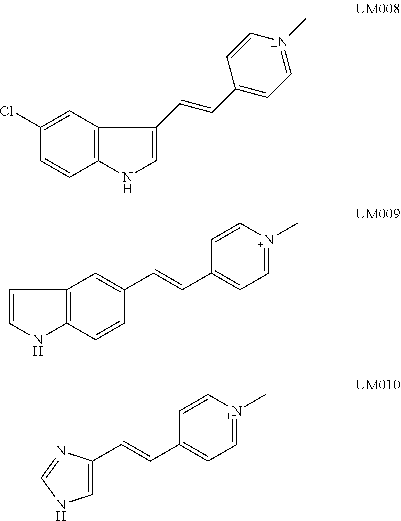

The compositions described herein can include one, two, three, or more compounds from the group UM001 through UM010.

In some examples, the composition can include a first selective fluorescent emitter selected from a first one of Formulas I through IX, and a second selective fluorescent emitter selected from a different one of Formulas I through IX. The first and second selective fluorescent emitters can both fluoresce at wavelengths above 400 nm when irradiated by an excitation radiation having a wavelength below 400 nm or below 410 nm. The first and second selective fluorescent emitter can fluoresce at different wavelengths.

Where at least two selective fluorescent emitters are used, the detecting step can include generating separate images using the fluorescence radiation emitted by each of the at least two selective fluorescent emitters. For example, where two selective fluorescent emitters are utilized, the emission wavelength (λ_{EM1}) of the first selective fluorescent emitter can be used to generate a first image and the emission wavelength (λ_{EM2}) of the second selective fluorescent emitter can be used to generate a second image by processing the same parent image. The first and second emission wavelengths can be different, i.e., λ_{EM1}≠λ_{EM2}.

The selective fluorescent emitters can be provided in solid form, in a solution, or in any other useful form. The composition can include a solvent. The solvent can be an organic solvent, an aqueous solvents, or both. Exemplary solvents include dimethyl sulfoxide (DMSO), methanol, phosphate-buffered saline (PBS), Dulbecco's phosphate-buffered saline (DPBS), cell grown medium, and mixtures thereof.

When provided in solid form, the selective fluorescent emitters can be held together by a binding agent. The solid composition of selective fluorescent emitters can be solubilized in an organic solvent, such as DMSO or methanol, prior to use. Prior to use or distribution, the solubilized selective fluorescent emitters can then be diluted in an aqueous solvent, such as a cell growth medium, PBS, DPBS, or a mixture thereof.

The concentration of the selective fluorescent emitters can range from 1 nM (nanomolar) to 10 M. For example, the composition could be a stock solution having a selective fluorescent emitter concentration from 1 mM to 100 mM, or a dilution having a selective fluorescent emitter concentration from 50-500 μM, or a further dilution having a selective fluorescent emitter concentration from 1 to 50 μM.

The invention also includes selective fluorescent emitters. The selective fluorescent emitters can be compounds of Formulas I through IX:

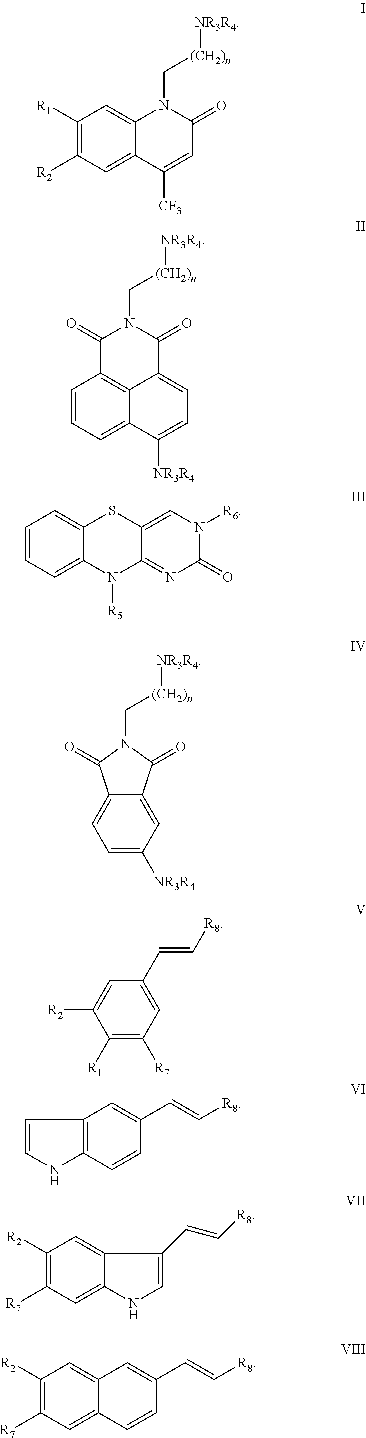

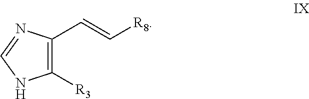

wherein:
R₁=OR₄ or NR₃R₄;
R₂=OR₄, NR₃R₄, Cl, F or H;
R₃=H, CH₃, C₂H₅, C₃H₇ or C₄H₉;
R₄=H, CH₃, C₂H₅, C₃H₇ or C₄H₉;
R₅=H or CH₂(CH₂)ₙNR₃R₄;
R₆=H or CH₂(CH₂)ₙNR₃R₄;
R₇=OR₄, NR₃R₄, Cl, F or H;
R₈=N-methylpyridinium or N-methyl-1,2,3,6-tetrahydropyridine; and
n=1, 2 or 3.

The inventive compounds can also be any of the more specific compounds or groups of compounds that are included in the compositions described herein.

The function and advantage of these and other embodiments of the present invention will be more fully understood from the examples described below. The following examples are intended to facilitate an understanding of the invention and to illustrate the benefits of the present invention, but are not intended to limit the scope of the invention.

### EXAMPLES

### Example 1

5-hydroxytryptamine (5HT, serotonin) is a classic neurotransmitter implicated in multiple emotional and behavioral disorders including depression. The serotonergic system is the subject of intense research aimed at understanding the basic mechanisms of disease and developing pharmaceutical interventions. Therefore, new tools that enable detailed, molecular level investigations of the serotonergic system are of great interest. Probes 1-4 (below) were designed around a carbostyril core.

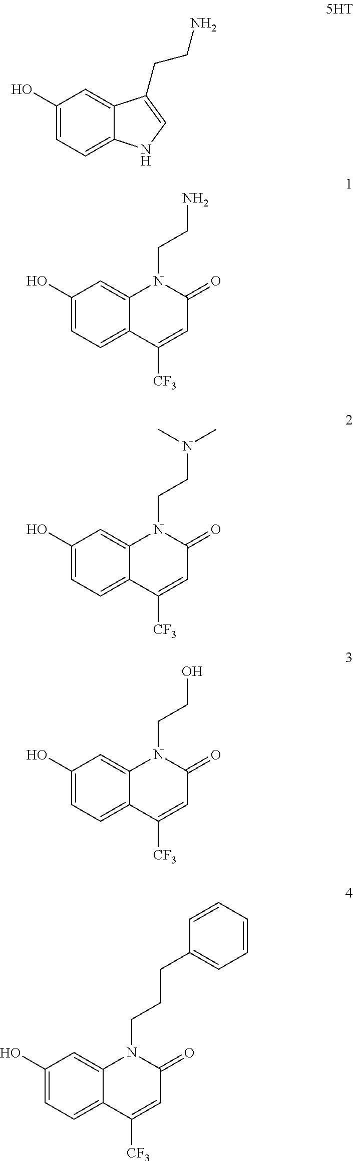

As shown below, 6-Methoxycarbostyril, 5, was synthesized as described by Fabian et al. Reaction of compound 5 with 1.2 equiv of 2-azidoethyl tosylate in DMF with K₂CO₃ as base produced intermediate azido compound 6. Reduction with PMe₃ in wet THF afforded the aminoethyl carbostyril, compound 7. Finally, removal of the methyl protecting group was achieved by reacting with 1.5 equiv of thiophenol with K₂CO₃.

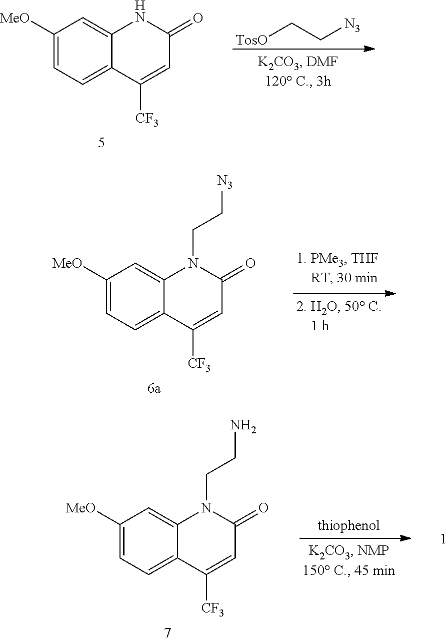

The synthesis of analogs 2-4 followed a similar route: reaction of 5 with chloroethylamine followed by deprotection of the 6-hydroxy group produced 2; 3 and 4 resulted from reaction of 5 with bromoethanol and bromopropylbenzene respectively. In all cases the N-linked isomer was isolated. All compounds are freely soluble in methanol; 1-3 are easily dissolved in phosphate-buffered saline (PBS) as well, while 4 is soluble only at dilute concentrations (<50 μM).

The optical properties of 1-4 were investigated in order to determine their suitability as fluorescent probes. Absorption spectra were obtained in neutral, acidic and basic solutions. In DPBS (pH 7.2) 1-4 exhibit absorption maxima between 350-355 nm with two prominent shoulders at 340 nm and 370 nm (FIG. 1). Addition of acetic acid effected a hypsochromic shift (λₘₐₓ=335 nm); the addition of triethylamine results in pronounced bathchromic (λₘₐₓ=370 nm) and hyperchromic shifts. The linear combination of the absorption spectra with excess acid and excess base nearly perfectly overlaps with the spectrum obtained at pH 7.2 (FIG. 1). Therefore, it was determined that at near neutral pH (between approximately 6.5-7.5pH), both protonated and unprotonated species exist at roughly equal concentrations.

The emission maxima for 1-4 was found to be ca. 480 nm in Dulbecco's phosphate-buffered saline (DPBS), representing a Stokes shift of 125 nm. The addition of excess base did not significantly shift the emission maxima. Thus, it was determined that in neutral and basic solutions, 1-4 emit from a deprotonated excited state species. Emission in acidic solutions is dominated by the deprotonated species as well. In the case of 3 and 4 a small peak at 380 nm was observed, which is likely due to photoemission from the protonated form of these molecules. While 5HT itself is fluorescent, single photon excitation of 5HT requires specialized optics and a UV excitation source (λ_{max,abs}=277 nm). Furthermore, the autofluorescence of biological samples is also quite high in the UV region. The longer wavelength absorption of 1-4 allows selective photoexcitation of these fluorophores in the presence of aromatic amino acid residues and nucleobases. The excitation and emission wavelengths of 1-4 are comparable to the commonly employed probes DAPI and Hoechst 33258 enabling the use of commercially available filter sets. In addition, the absorption spectrum possesses a broad shoulder extending past 400 nm which facilitates excitation by standard 405 nm diode lasers as well.

Mixed cell cultures of neurons and astroglia isolated from the brain stems of E14 chick embryos were exposed to solutions of 1-4. Serotonergic cells are clustered in the raphe nucleus (RN) located along the midline of the brain stem; they project into other regions of the brain including the hypothalamus, the olfactory bulb, cortex and cerebellum. It is believed that if 1-4 were effective as serotonin mimics, they would exhibit affinities for the serotonergic cells present in the RN. The inherent fluorescence of the carbostyril core enabled direct detection of their cellular uptake utilizing a microwell plate reader (λₑₓ=355 nm, λₑₘ=444 nm). A high emission response was observed for 1 and to a lesser extent, 4, while 2 and 3 did not differ significantly from the controls (Control A: cells alone, Control B: wells treated compound only, then rinsed).

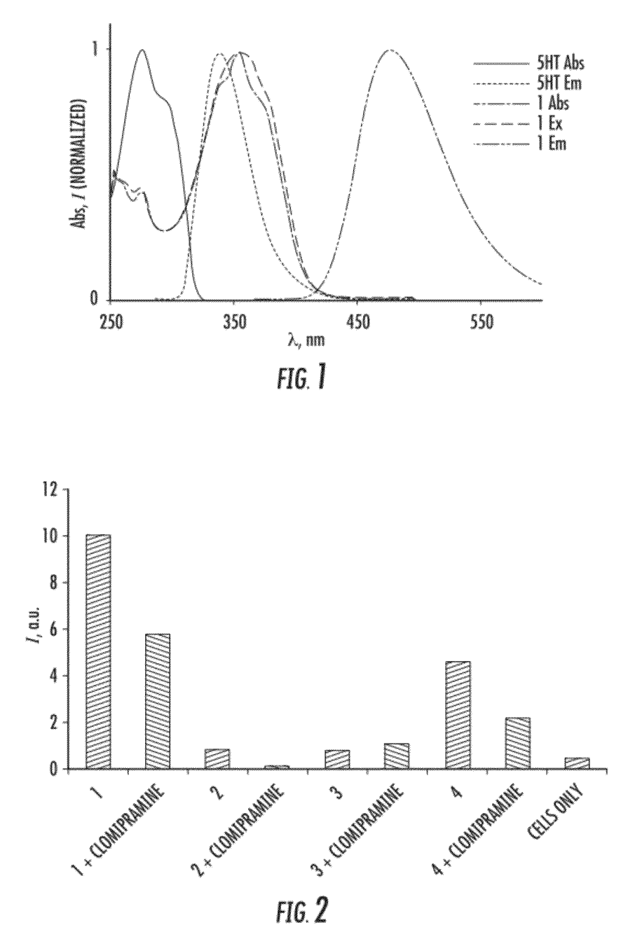

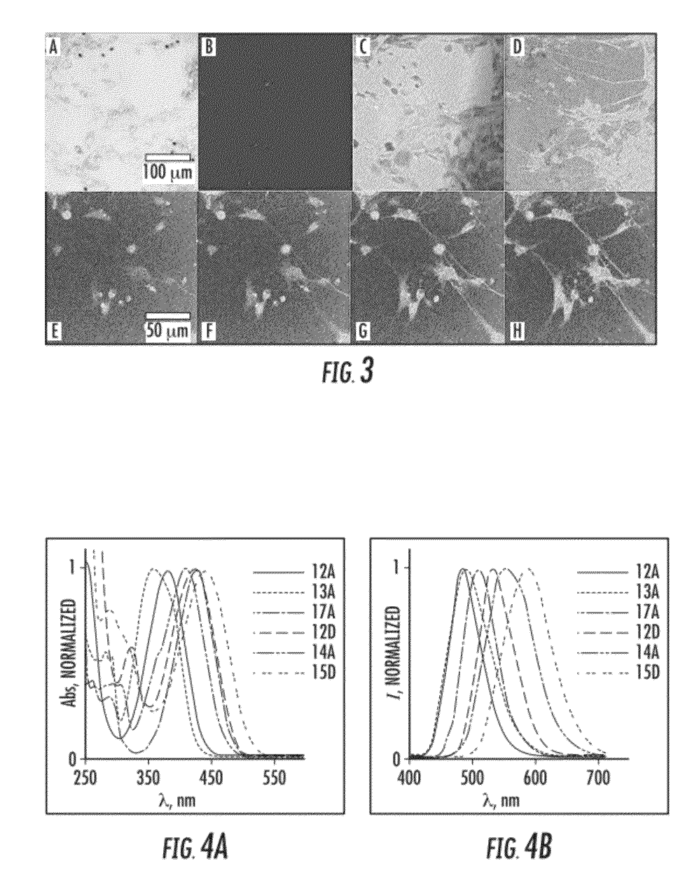

It was believed that several modes of interaction are possible between the fluorescent probes and the cultured cells including specific interactions such as active transport via neurotransmitter transporters, or binding to neurotransmitter receptors, as well as non-specific interactions such as insertion into the cell membrane. As shown in FIG. 2, pre-treatment of the cell cultures with solutions of Clomipramine, a serotonin reuptake inhibitor (SRI), significantly reduced the uptake of 1 and 4. This demonstrates that at least one mode of fluorophore-cell interaction involves serotonin transporters.

The lack of response of 2 relative to 1 is somewhat surprising as they differ only by two methyl groups. The observed uptake of 4 was also unexpected as it lacks the amine functionality typical of most neuroactive compounds. While not wishing to be bound by theory and not necessary to use the invention disclosed herein, it is believed that π-π interactions may allow 4 to be a substrate for SERT.

While based on the microwell plate assay above, compound 1 showed differential affinities for cultured brainstem cells in the absence and presence of an SRI, further evaluations were conducted to determine the fate of the fluorescent probe. In other words, whether the probe localized to the cell membrane, implying binding to transporters or receptors, or whether the probe was internalized, implying active transport into the cytosol. Neurons and astroglia isolated from E14 chick brainstems were grown on poly-L-lysine and laminin treated coverslips affixed to 35 mm culture dishes enabling live imaging of the cells in culture media. Excitation was achieved with a 405 nm diode laser; emission wavelengths were selected by adjusting the window of a prism to 450 through 600 nm. Cells were exposed to the probe solution for one minute, the media was then changed and additional images were captured. As is evident in FIG. 3, compound 1 bound to and illuminated most healthy cells. Z-stacks of the cells were obtained (FIG. 3, bottom row) and found that compound 1 was indeed localized internally after only one minute of exposure. During the course of the imaging experiments, some photobleaching was observed. However, compound 1 was sufficiently stable to capture multiple images over 10 mins without the use of antifade reagents or solutions.

Based on the foregoing screening experiments to evaluate probe activities with cultured neurons and astroglia in the presence and absence of the SRI clomipramine, it was determined that two classes of probes, represented by compounds 1 and 4, can function as fluorescent analogs of serotonin (5HT). Confocal images of cells exposed to compound 1 reveal that this probe is localized to the interior of both astroglia and neurons.

### Example 2

A family of stilbazolium dyes was synthesized, characterized by optical spectroscopy and screened for uptake in cultured brainstem and cerebellum cells isolated from e19 chick cells. Pretreatment of cells with indatraline, a monoamine reuptake inhibitor, allowed identification of compounds that may interact with monoamine transporters. Two structurally related, yet spectrally segregated, probes, (E)-1-Methyl-4-[2-(2-naphthalenyl)ethenyl]-pyridinium iodide (NEP+, 13A) and (E)-4-[2-(6-hydroxy-2-naphthalenyl)ethenyl]-1-methyl-pyridinium iodide (HNEP+, 14A, UM006), were selected and further investigated using HEK-293 cells selectively expressing dopamine, norepinephrine or serotonin transporters. HNEP+ was selectively accumulated via catecholamine transporters, with the norepinephrine transporter (NET) giving the highest response; NEP+ was not transported, though possible binding was observed. The alternate modes of interaction enable the use of NEP+ and HNEP+ to image distinct cell populations in live brain tissue explants. The preference for HNEP+ accumulation via NET was confirmed by imaging uptake in the absence and presence of desipramine, a norepinephrine reuptake inhibitor.

### Family of Potential Selective Fluorescent Emitters

A 32 member family of cationic arylene-vinylene emitters (actually potential emitters) was synthesized via Knoevenagel condensation of either a N-methyl-picolinium iodide or 1,4-dimethylquinolium iodide (A-D) with an aryl aldehyde (11-18), all of which are shown below:

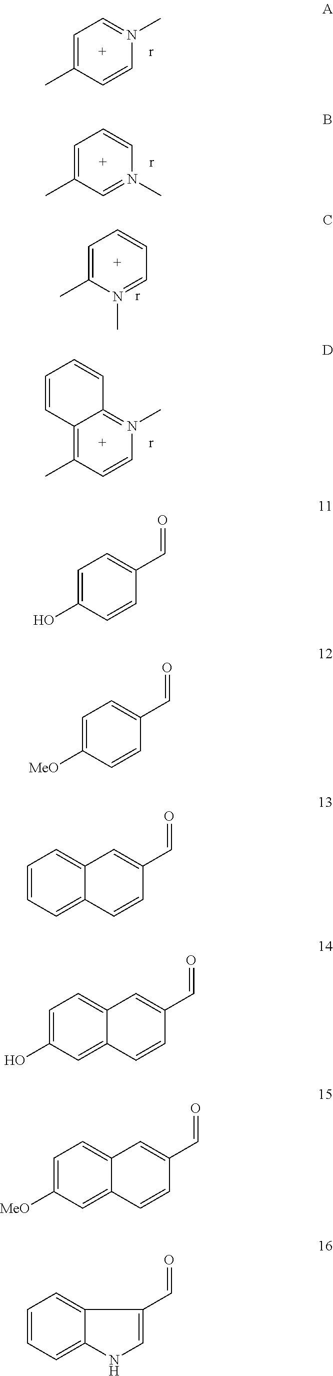

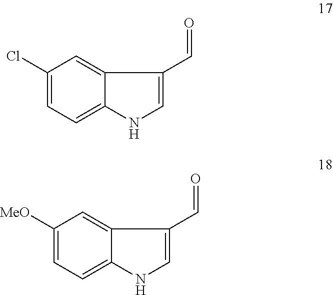

An exemplary Knoevanagel condensation of constituent A and constituent 11 to produce compound 11A (which is also referred to as UM005) is shown below:

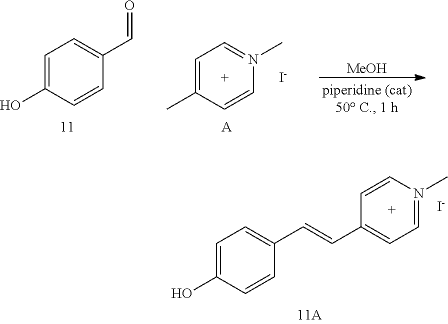

Compounds were isolated as brightly colored crystalline solids by filtration, purified by crystallization and selected emitters were characterized by 1H-NMR, 13C-NMR, IR, HRMS, UV-vis and fluorescence spectroscopy.

### Optical Spectroscopy

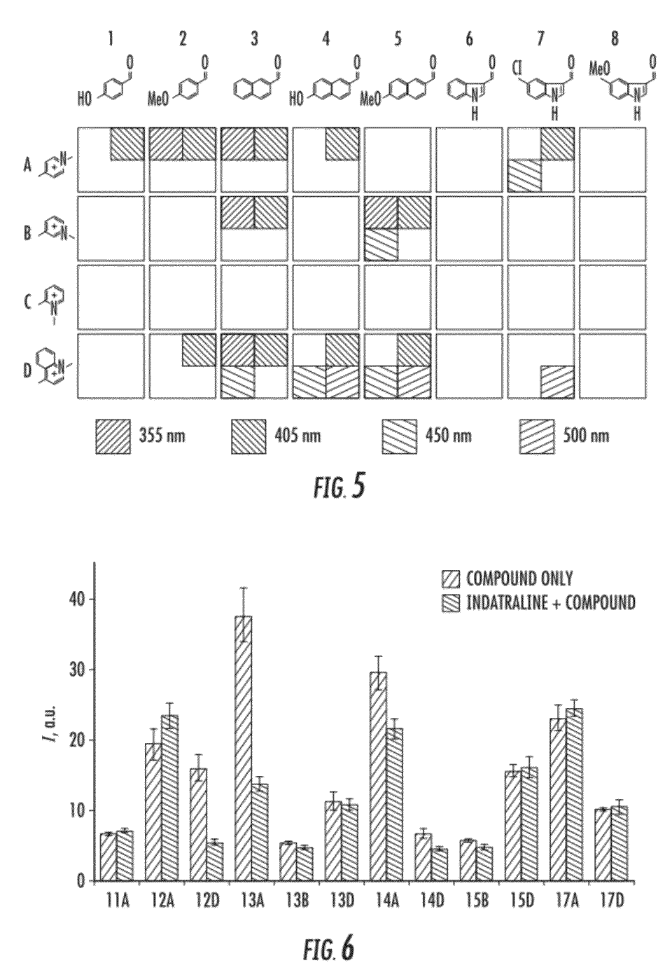

UV-vis spectroscopy was conducted to evaluate the absorption and emission spectra for each of the thirty-two emitters. FIG. 4 shows normalized absorption (FIG. 4A) and emission spectra (FIG. 4B) of selected library members in 10 μM solutions. Absorption maxima vary from the near UV to blue; however, all emitters are compatible with 405 nm laser exciation. Emission ranges span the entire visible spectrum with large (150+ nm) Stokes shifts for several emitters.

Or particular interest, the data reveals absorption maxima ranging from 358 nm for 12A to 441 nm for 15D. As shown in FIG. 4, the peaks lack vibronic structure, characteristic of intramolecular charge transfer absorption expected for donor-acceptor systems. Emission maxima spanned much of the visible spectrum and range from 475 nm for 12A, to 623 nm for 15D. The emitters possess large Stokes shifts of 120 to 165 nm. Compounds containing the quinolium functionality, D, exhibit longer absorption and emission wavelengths than the corresponding pyridinium analogs, e.g., 12A vs 12D (λmax, em=490 and 540 nm, respectively). Benzo-fusion produces a red shift in the case of 12A vs 15A, as well. Introduction of electron donating groups also results in a bathchromic shift as in the case of 14A, the hydroxy substituted homolog of 13A shifting the emission maxima approximately 80 nm. Epifluorescent imaging of the emitters can be accomplished using conventional filter sets such as those for DAPI and GFP. All of the dyes can be excited using a 405 nm laser, enabling confocal imaging without the need for UV specific optics. The broad range of emission wavelengths allows for selective detection of appropriately paired emitters (i.e., 12A or 13A in combination with 14A or 15D). Molar absorptivities (ε) ranged from 30,000 to 55,000 M⁻¹cm⁻¹ with quantum yields of photoemission (Φₑₘ) ranging from 0.20 to 0.01. While emitters with low Φₑₘ are of limited utility, the overall brightness (ε·Φₑₘ) of emitters utilized in imaging experiments (13A and 14A, see below) compares favorably with existing fluorescent probes such as acridine orange, Hoescht 33258 and DAPI.

### Cellular Uptake and Inhibition Studies

The native fluorescence of the emitters enables direct detection of their uptake in dissociated e19 chick brain cells in a convenient 96-microwell plate format. This method enables screening for activity against multiple cell types and transporters including MATs and OCTs without the use of radioactive compounds. Solutions of 11A-18D were added to cell culture media to produce an emitter concentration of 100 μM. The incubation time used was 10 min. The fluorphore solution was removed, fresh media added and the plate immediately analyzed by microwell plate reader. Given the broad range of absorption and emission wavelengths, excitation varied from 355 nm to 500 nm with emission collected at +100 nm to +150 nm. Twelve of the thirty-two compounds screened were identified as hits in this initial assay (FIG. 5) based on the increase in emission intensity compared to untreated cells (values of 3× control were considered hits). FIG. 5 shows whether there was uptake of each of the 32 compounds by dissociated whole brain cells harvested from e19 chicks and whether the treated brain cells exhibited a Stokes shift of at least 100 nm for excitation radiation at 365 nm (top left), 405 nm (top right), 450 nm (bottom left) and 500 nm (bottom right).

Components A and D are well represented, while no compounds possessing C, 16 or 18 were detected in this screen. This may be due to a combination of factors including low uptake, low brightness or high cytotoxicity resulting in cell detachment and removal during the washing step.

From this initial assay, it was possible to determine whether a compound associates with the cultured cells; however, it was not clear whether a particular compound was actively transported into cells or simply interacts with the cell membrane. The monoamine transporter (MAT) reuptake inhibitor, indatraline, was utilized to provide some insight into what mechanisms of cellular uptake are at play. Dissociated brain cells were pretreated with indatraline followed by each of the twelve emitters identified from the results shown in FIG. 5.

As shown in FIG. 6, the emission response of several probes was affected by indatraline pretreatment, including 12D, 13A, 14A and 14D, suggesting that at least one mode of cellular uptake for these compounds is via a MAT. In contrast, three probes, 12A, 15D and 17A maintained high responses even after indatraline pretreatment, which indicates these compounds either simply interact with the cell membrane, or are accumulated via other transporters, i.e. organic cation transporters (OCTs). The remaining compounds possessed weak responses or low overall intensity.

### Live Cell and Tissue Imaging

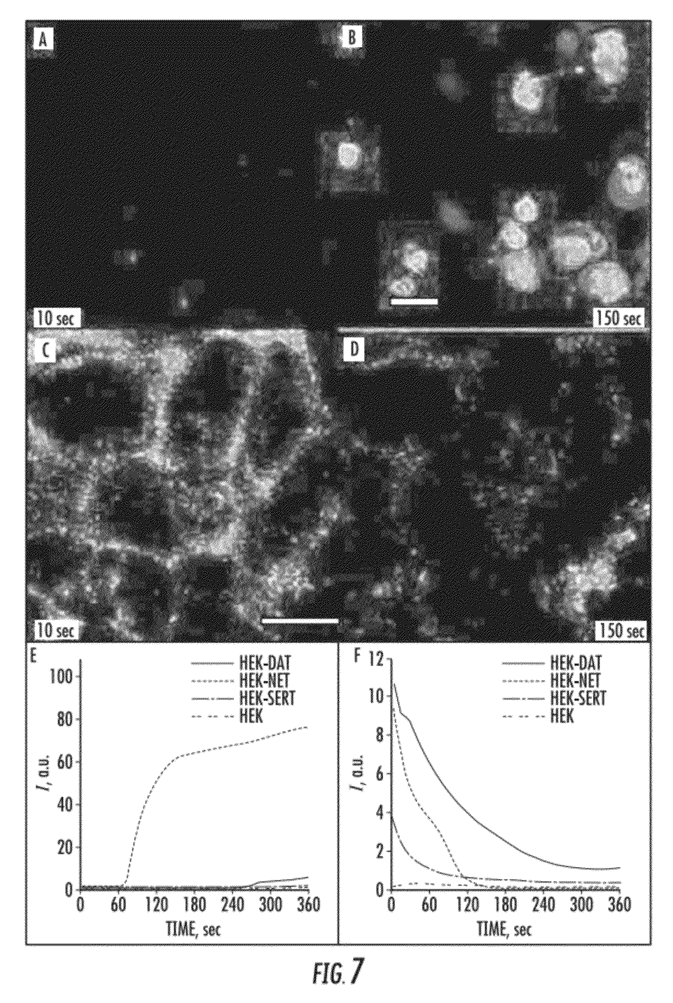

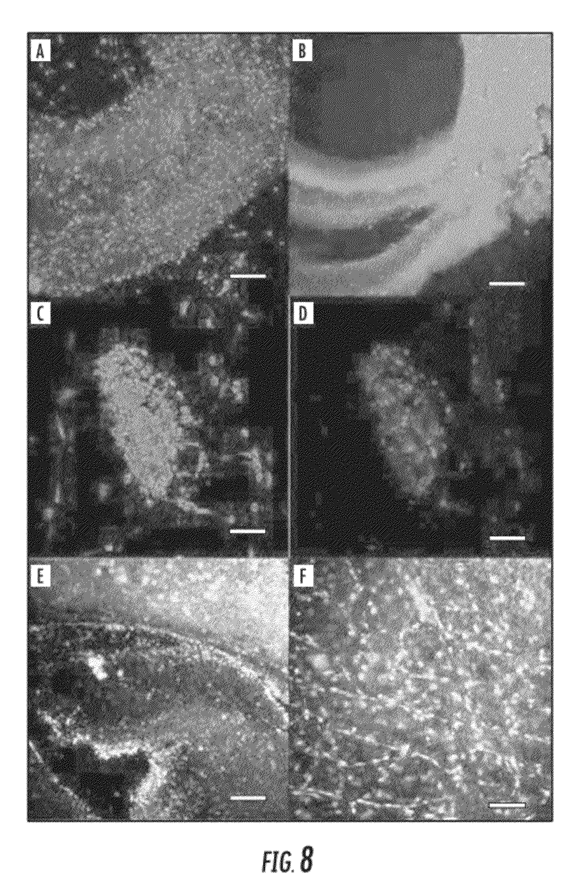

The two brightest compounds inhibited by indatraline, 13A and 14A, were selected for further characterization in live brain explants as well as cell lines expressing specific MATs in order to evaluate their use as probes of MAT function. HEK-293 cells stably expressing hDAT, hNET and hSERT were provided by Prof. R. Blakely (See, e.g., J. W. Schwartz, R. D. Blakely and L. J. DeFelice, J. Biol. Chem., 2003, 278, 9768.) and cultured on 35 mm dishes with a coverslip imaging window for confocal microscopy. Baseline images for each cell type were taken to establish the level of cell autofluorescence, then stock solutions of compounds 13A and 14A were introduced to produce final concentrations ranging from 10 μM to 100 μM. Laser intensity and gain were not altered for subsequent images captured at 10 sec intervals. Representative images are shown in FIG. 7 together with intensity profiles captured from cell bodies.

FIG. 7 shows the contrasting behavior of 13A and 14A towards HEK293 cells expressing DAT, NET or SERT. As shown in FIGS. 7A (10 second after introduction of 14A) and 7B (150 second after), time-lapse fluorescence microscopy shows that 14A preferentially accumulates in cells expressing NET. By contrast, as shown in, FIGS. 7C (10 seconds after introduction of 13A) and 7D (150 second after), 13A did not accumulate in cells expressing DAT, NET or SERT, and emission intensity decreased with time due to photobleaching. FIGS. 7E and 7F show time-dependent intensity plots for 14A and 13A, respectively.

The time lapse images reveal that 14A is rapidly accumulated by HEK-hNET and to a lesser extent by HEK-hDAT. Based on the time dependent intensity profiles, the response towards HEK-hSERT cells does not differ substantially from the control untransfected HEK cells. Inspection of images obtained for DAT and SERT cells reveals that 14A may be binding to these transporters as fluorescent halos were observed; however, transport does not appear to occur in the case of SERT and occurs at a much slower rate (and delayed onset) in the case of DAT.

By contrast, 13A does not appear to be transported via DAT, NET or SERT. Rather, 13A may bind to cells expressing these transporters. However, a slow loss of fluorescence due to photobleaching was observed, rather than an increase of emission intensity within the cell bodies (FIG. 7). The scale bars for FIG. 7 are 10 μm.

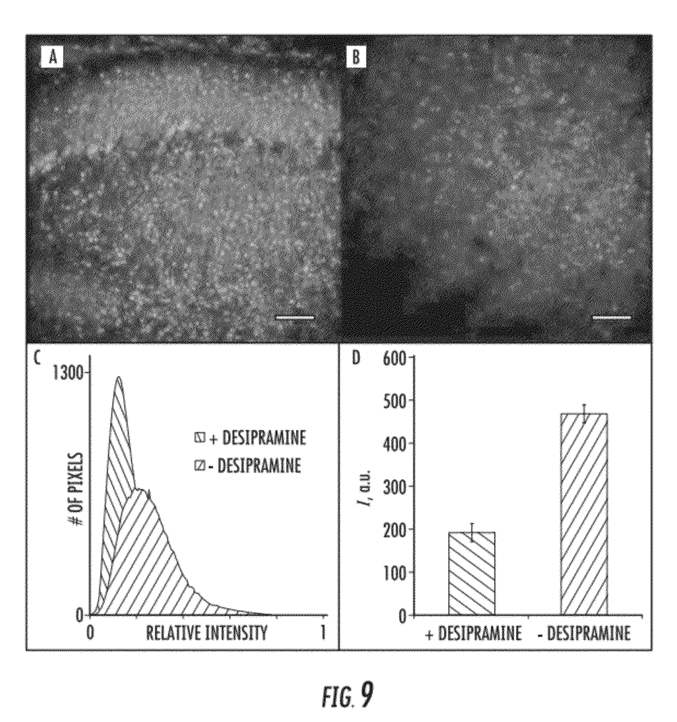

Compounds 13A and 14A were subsequently examined using live brainstem and cerebellum sections from e19 chicks to determine the distribution of the compounds in a complex, multicellular environment. FIGS. 8 (A,B) shows confocal microscopy images of cerebellum sections treated individually with 13A and 14A, respectively. The images show 13A appears to be more widely distributed than 14A, though both dyes are limited to discreet cell populations tracing the cerebellar gyri. This distribution is consistent with the behavior of these probes towards MAT-expressing HEK293 cells. The wider distribution of 13A may be due to binding to multiple transporter types, while 14A may be limited to NET or DAT expressing catecholaminergic cells. The spectral segregation of 13A and 14A enables their simultaneous use to identify cells that differentially accumulate each probe. Image overlays from 100 μm sagittal (FIG. 8A) and transverse (FIG. 8B) sections reveal that both probes are associated with cells of chick mesencephalon and metencephalon.

The fluorophores were excited by laser at 405 nm. The emission of 13A (in green) was collected between 425 and 500 nm, while the emission of 14A (in red) was collected from 575 to 650 nm with areas of overlap appearing as yellow. Compound 13A is widely distributed in both the cerebellum and pons, while compound 14A appears in distinct cell groupings along the cerebellar gyri and around the midline of the brainstem especially in multiple tract-like structures. The chick brainstem possesses dense clusters of chatecholaminergic and serotonergic cells that project into the cerebellum and front brain. The images show both perikarya and fibers stained with 14A in this region with several discreet cell clusters brightly illuminated (FIG. 8C). The scale bars for FIG. 8 are as follows: 8A & 8B=100 μm; C & D=10 μm; and E & F=50 μm).

Pretreatment of brain sections with desipramine, a norepinephrine reuptake inhibitor, limited the accumulation of 14A (FIG. 9), which is consistent with the results observed for hNET-expressing HEK-293 cells. Visual inspection of the images reveals a distinct difference between desipramine treated (FIG. 9B) and untreated (FIG. 9A) tissue, which correlates with image analysis. While individual cell bodies and processes are visible in cells exposed to 14A, pretreatment with desipramine limits the overall emission intensity (FIG. 9D) and the lack of accumulation to discreet structures or cells is evident in intensity histograms (FIG. 9C).

### SUMMARY

A library of cationic stilbazolium emitters was synthesized in an attempt to identify selective fluorescent emitters suitable as imaging agents and reporters of transporter function. The results demonstrated that MATs are capable of transporting compounds that mimic certain aspects of the parent substrates. It appears the aryl cation in this library serves to replace the ethylamine functionality of the biogenic amines. Several compounds were found to associate with whole brain cell cultures in the absence of indatraline, suggesting that the stilbazolium core may be a versatile substrate for MATs. The necessity for dyes possessing high brightness (ε·Φₑₘ) is also apparent, as only 14A (also referred to at UM006) and 13A proved useful for in vitro and ex vivo imaging. Time dependent imaging of 14A reveals a preference for NET, similar to ASP+. However in contrast to ASP+, 14A does not appear to be transported via SERT making 14A a selective catecholaminergic emitter with a high level of discrimination between DAT and NET.

The results also demonstrate that subtle changes in structure lead to pronounced differences in behavior towards MATs: no accumulation of 13A was observed for DAT, NET or SERT expressing HEK293 cells, though 13A may interact or bind to the transporters. This contrasting behavior between 13A and 14A is also apparent in live brain tissue imaging: while 14A targets specific cell groupings, most likely noradrenergic, 13A exhibits broad distribution.

Finally, it has been demonstrated that 14A can be used to image noradrenergic cells in live tissue samples and directly assess the function of NET in the absence and presence of reuptake inhibitors. Given the complex modes of transporter regulation, the ability to monitor transporter function in tissue explants (or in vivo) may lead to improved assays and identification of new classes or improved selectivity of reuptake inhibitors.

### Example 3

Selective uptake via human norepinephrine transporter (hNET or simply NET) has been demonstrated for (E)-4-[2-(6-hydroxy-2-naphthalenyl)ethenyl]-1-methyl-pyridinium iodide (HNEP+, 14A, UM006, structure below).

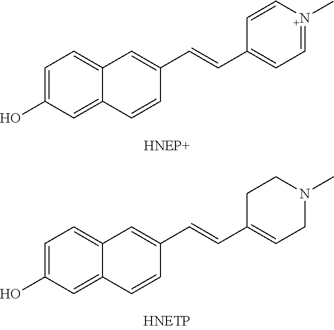

The selectivity of HNEP+ enables identification of noradrenergic cells in vitro, ex vivo and in vivo. Furthermore, the activity of selective norepinephrine reuptake inhibitors (NRIs) can be assessed in live brain tissue preparations using HNEP+ as a reporter of transporter function. This demonstrates several applications of HNEP+ are viable:

1. Use in high-throughput functional assays (as opposed to binding or competitive assays) for screening of novel pharmaceutical compounds targeting NET (such as antidepressants and related pharmacotherapies).

2. Use as a basic research tool for assessing the location and activity state of NET in cell culture, tissue explants as well as live organisms (e.g. Danio rerio, C. Elegans).

In addition, it has been discovered that reduction of HNEP+ (UM006) results in the formation of HNETP (UM007). This provides a profluorescent option for screening pharmaceutical targets in vivo, which allows for the assessment of bioavailability of compounds in model organisms. In particular, the long wavelength absorption and emission of HNEP+ are not present in HNETP due to the loss of conjugation and the cationic pyridinium functionality. However, the action of monoamine oxidases (MAOs) convert HNETP to HNEP+ in cell culture. This reflects two distinct processes: accumulation of HNETP in the cells and the metabolism of HNETP to HNEP+ by MAOs. Inhibitors of MAO are important for several diseases including Parkinsons Disease as well as hypertensive disorders.

Applications in drug screening of MAO inhibitors both in vitro and in vivo are envisaged. For example, HNETP (UM007) can be injected or otherwise introduced to target tissue in the presence of a potential MAO inhibitor. Because HNETP does not fluoresce, if no fluorescence is present upon irradiation with an excitation wavelength for HNEP+ (UM006), the MAO inhibitor is working. If fluorescence occurs, it means the MAOs acted on the HNETP (UM007) to form the fluorescent reduction product HNEP+ (UM006).

### Example 4

Stock solutions of the compounds shown below (UM001-UM005, 8, 9 & 12A from Example 2) were used to produce compositions with final concentrations ranging from 10 μM to 100 μM. The compositions were then contacted with dissociated e19 chick brain cells, irradiated with a 405 nm laser, and imaged using confocal microscopy.

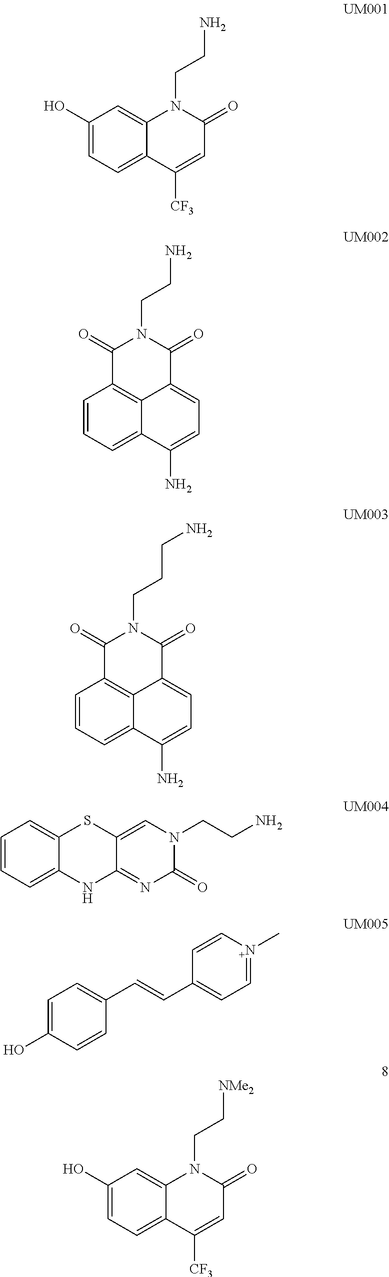

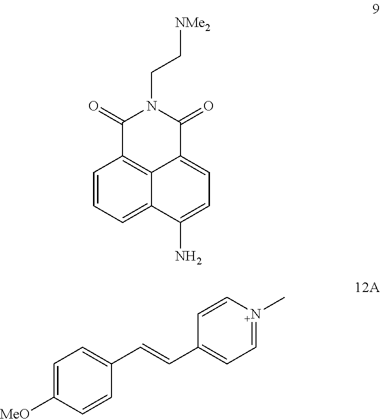

The results demonstrated that compounds UM001-UM005 & 12A are accumulated in dissociated chick brain cells in the absence of monoamine reuptake inhibitors (MRIs) as determined by the increase in fluorescence in a microwell plate assay and/or live cell imaging. By contrast, dialkylated derivatives 8 and 9 demonstrated little or no uptake. There is some evidence that compounds 8 and 9 may be binding to transporters, but it is believed that the transport is hindered due to the lipophilic groups. Based on the foregoing, UM001-UM005 & compound 12A can be useful selective fluorescent emitters.

It is to be understood that while the invention in has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

We claim:

1. A method of imaging comprising:
contacting a specimen with a composition comprising a selective fluorescent emitter selected from Formulas I through IX; and
irradiating said specimen with an excitation wavelength, wherein Formulas I through IX are: 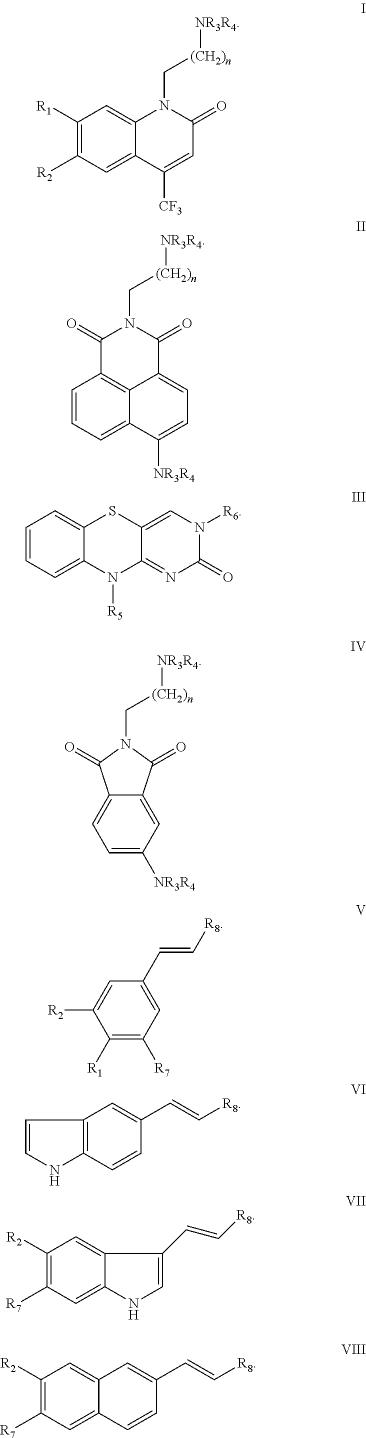 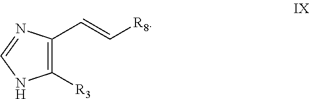
wherein:
R₁=OR₄ or NR₃R₄;
R₂=OR₄, NR₃R₄, Cl, F or H;
R₃=H, CH₃, C₂H₅, C₃H₇ or C₄H₉;
R₄=H, CH₃, C₂H₅, C₃H₇ or C₄H₉;
R₅=H or CH₂(CH₂)ₙNR₃R₄;
R₆=H or CH₂(CH₂)ₙNR₃R₄;
R₇=OR₄, NR₃R₄, Cl, F or H;
R₈=N-methylpyridinium or N-methyl-1,2,3,6-tetrahydropyridine; and
n=1, 2 or 3.

2. The method of imaging according to claim 1, further comprising:
detecting fluorescence from said specimen to generate an image.

3. The method of imaging according to claim 2, wherein said detecting step comprises obtaining a camera imaging of said specimen.

4. The method of imaging according to claim 2, wherein said detecting step comprising filtering out electromagnetic radiation below 400 nm.

5. The method of imaging according to claim 1, wherein said selective fluorescent emitter emits fluorescence at a wavelength above 400 nm when irradiated at said excitation wavelength.

6. The method of imaging according to claim 1, wherein said excitation wavelength comprises electromagnetic radiation having a wavelength below 400 nm.

7. The method of imaging according to claim 1, wherein said irradiating step comprises irradiating with a monochromatic electromagnetic radiation source.

8. The method of imaging according to claim 7, wherein said monochromatic electromagnetic radiation source comprises radiation having a wavelength below 400 nm.

9. The method of imaging according to claim 7, wherein said monochromatic electromagnetic radiation source is a diode laser.

10. The method of imaging according to claim 1, wherein said selective fluorescent emitter comprises a compound selected from the group consisting of Formulas I, II, III and IV, and wherein R₃=H or CH₃ and R₄=H or CH₃.

11. The method of imaging according to claim 10, wherein R₃=R₄=H.

12. The method of imaging according to claim 10, wherein R₂=H, R₅=H and R₆=CH₂(CH₂)ₙNR₃R₄.

13. The method of imaging according to claim 10, wherein n=1 or 2.

14. The method of imaging according to claim 1, wherein said selective fluorescent emitter comprises a compound selected from the group consisting of Formulas V, VI, VII, VIII and IX, and wherein R₁=OH, OCH₃, NH₂, NHCH₃ or N(CH₃)₂, and R₃=H or CH₃.

15. The method of imaging according to claim 1, wherein said selective fluorescent emitter comprises a compound of Formula V, wherein R₁=OH or OCH₃ and R₂=R₇=H.

16. The method of imaging according to claim 1, wherein said selective fluorescent emitter comprises a compound of Formula VII or VIII, wherein R₇=OH, OCH₃, NH₂, NHCH₃ or N(CH₃)₂ and R₂=H.

17. The method of imaging according to claim 1, wherein said selective fluorescent emitter comprises a compound of Formula VII or VIII, wherein R₂=H and R₇=OH, OCH₃, or H.

18. The method of imaging according to claim 1, wherein said selective fluorescent emitter comprises a compound of Formula IX, wherein R₃=H or CH₃.

19. The method of imaging according to claim 1, wherein said selective fluorescent emitter comprises at least one of the following compounds:

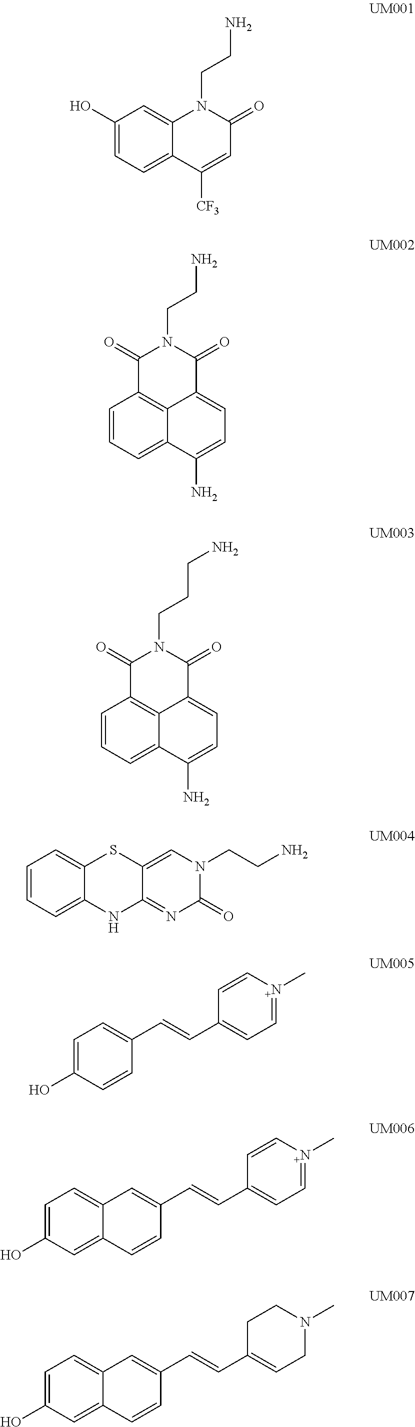

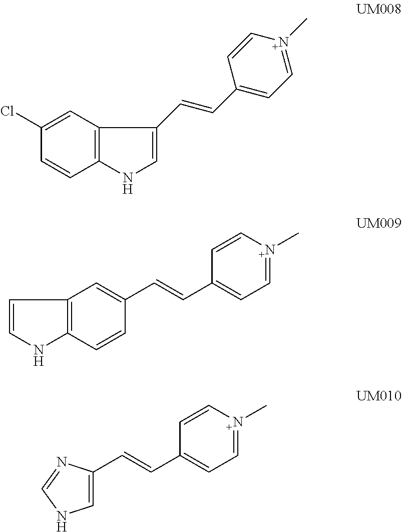

20. The method of imaging according to claim 1, wherein said composition comprises a first selective fluorescent emitter selected from a first one of Formulas I through IX, and a second selective fluorescent emitter selected from a different one of Formulas I through IX.

21. The method of imaging according to claim 20, wherein said first and said second selective fluorescent emitter that fluoresce at wavelengths above 400 nm when irradiated by excitation radiation having a wavelength below 400 nm, and wherein said first and said second selective fluorescent emitters fluoresce at different wavelengths.

22. The method of imaging according to claim 21, wherein said detecting step comprises generating an image using fluorescence radiation emitted by said first selective fluorescent emitter and generating a second image using fluorescence radiation emitted by said second selective fluorescent emitter.

23. A composition, comprising:
a selective fluorescent emitter selected from Formulas I through IX, wherein Formulas I through IX are: 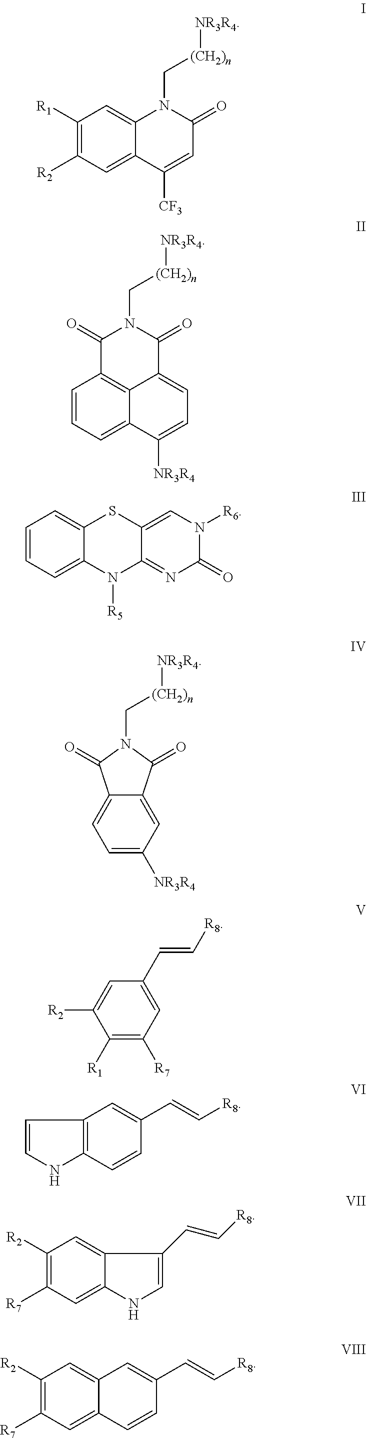 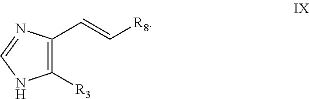
wherein:
R₁=OR₄ or NR₃R₄;
R₂=OR₄, NR₃R₄, Cl, F or H;
R₃=H, CH₃, C₂H₅, C₃₋H₇ or C₄H₉;
R₄=H, CH₃, C₂H₅, C₃H₇ or C₄H₉;
R₅=H or CH₂(CH₂)ₙNR₃R₄;
R₆=H or CH₂(CH₂)ₙNR₃R₄;
R₇=OR₄, NR₃R₄, Cl, F or H;
R₈=N-methylpyridinium or N-methyl-1,2,3,6-tetrahydropyridine; and
n=1, 2 or 3.

24. The composition according to claim 23, further comprising a solvent selected from the group consisting of an organic solvent, an aqueous solvent, or both.

25. The composition according to claim 23, wherein a concentration of said selective fluorescent emitter in said composition is 1 nM to 10 M.

26. The composition according to claim 23, wherein said selective fluorescent emitter comprises a first compound selected from a first one of Formulas I through IX, and a second compound selected from a different one of Formulas I through IX.

27. The composition according to claim 26, wherein said first and said second compound fluoresce at wavelengths above 400 nm when irradiated by excitation radiation having a wavelength below 400 nm, and wherein said first and said second compound fluoresce at different wavelengths.

28. A compound according to at least one of Formulas I through IX, wherein Formulas I through IX are: 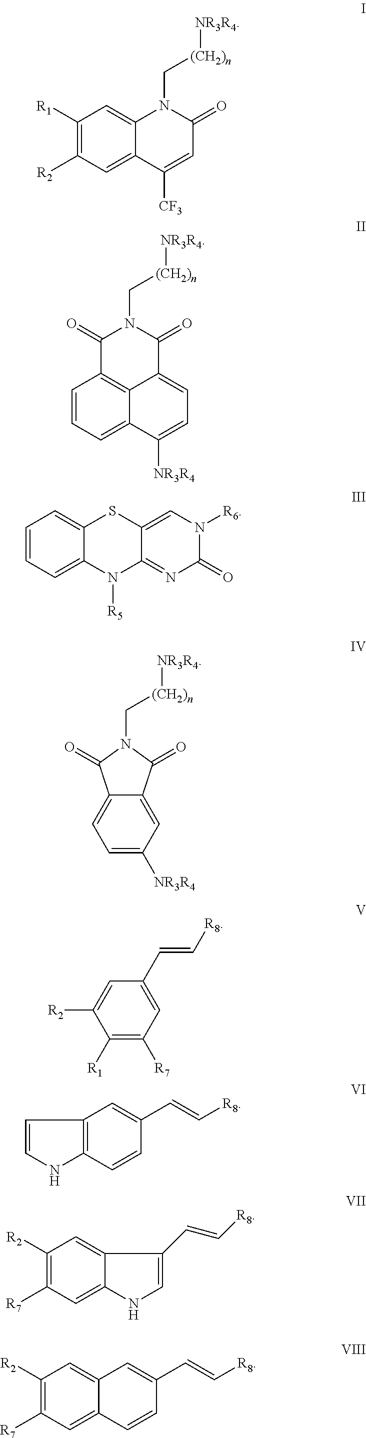 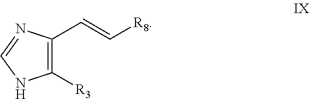
wherein:
R₁=OR₄ or NR₃R₄;
R₂=OR₄, NR₃R₄, Cl, F or H;
R₃=H, CH₃, C₂H₅, C₃H₇ or C₄H₉;
R₄=H, CH₃, C₂H₅, C₃H₇ or C₄H₉;
R₅=H or CH₂(CH₂)ₙNR₃R₄;
R₆=H or CH₂(CH₂)ₙNR₃R₄;
R₇=OR₄, NR₃R₄, Cl, F or H;
R₈=N-methylpyridinium or N-methyl-1,2,3,6-tetrahydropyridine; and
n=1, 2 or 3.

29. A compound according to claim 28, wherein said compound of Formulas I-IX is: [chemical structure]
